(19) United States
(12) Patent Application Publication
(10) Pub. No.: US 2015/0001505 A1
(43) Pub. Date: Jan. 1, 2015

(54) CYCLOBUTANE GROUP-CONTAINING COMPOUND AND ORGANIC ELECTROLUMINESCENCE DEVICE INCLUDING THE SAME

(71) Applicant: SAMSUNG DISPLAY CO., LTD., Yongin-City (KR)

(72) Inventor: Hisayuki KAWAMURA, Yokohama (JP)

(21) Appl. No.: 14/315,445

(22) Filed: Jun. 26, 2014

(30) Foreign Application Priority Data
Jun. 28, 2013 (JP) 2013-135968

(51) Int. Cl. H01L 51/00

(52) U.S. Cl. CPC H01L 51/005; H01L 51/506
USPC 257/40; 558/426

(57) ABSTRACT

A cyclobutane group-containing compound and an organic EL device, the compound being represented by following Formula 1:

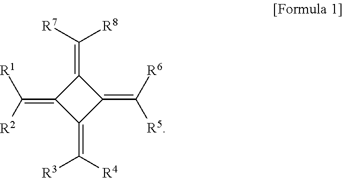

### CROSS-REFERENCE TO RELATED APPLICATION

Japanese Patent Application No. 2013-135968, filed on Jun. 28, 2013, in the Japanese Patent Office, and entitled: “Cyclobutane Derivatives and Organic Electroluminescence Device Including The Same,” is incorporated by reference herein in its entirety.

### BACKGROUND

1. Field

Embodiments relate to a cyclobutane group-containing compound and an electroluminescence device including the same.

2. Description of the Related Art

In recent years, organic electroluminescence (EL) displays (that are one type of image displays) have been actively developed. Unlike a liquid crystal display and the like, the organic EL display is a self-luminescent display in which holes and electrons injected from an anode and a cathode recombine in an emission layer to thus emit lights from a light-emitting material (including an organic compound of the emission layer), thereby performing display.

An example of an organic electroluminescence device (hereinafter referred to as an organic EL device) may include an organic EL device that includes a anode, a hole transport layer on the anode, an emission layer on the hole transport layer, an electron transport layer on the emission layer, and a cathode on the electron transport layer. Holes injected from the anode may be injected into the emission layer via the hole transport layer. Electrons may be injected from the cathode, and then injected into the emission layer via the electron transport layer. The holes and the electrons injected into the emission layer may be recombined to generate excitons within the emission layer. The organic EL device may emit light generated by radiation and deactivation of the excitons. The organic EL device may be changed in various forms.

### SUMMARY

Embodiments are directed to a cyclobutane group-containing compound and an electroluminescence device including the same.

The embodiments may be realized by providing a cyclobutane group-containing compound represented by following Formula 1:

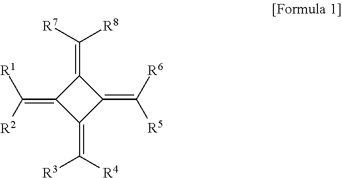

wherein, in Formula 1, R¹ to R⁸ are each independently a substituted or unsubstituted aryl group having 6 to 20 carbon atoms, a substituted or unsubstituted heteroaryl group having 5 to 20 carbon atoms, or a cyano group.

At least one of R¹ to R⁸ may include the substituted aryl group having 6 to 20 carbon atoms or the substituted heteroaryl group having 5 to 20 carbon atoms, and the substituted aryl group having 6 to 20 carbon atoms or the substituted heteroaryl group having 5 to 20 carbon atoms may be substituted with an electron-withdrawing group.

The electron withdrawing group may include a fluorine atom.

Two to four of R¹ to R⁸ may be cyano groups.

The cyclobutane group-containing compound may have a rotational symmetry structure of order 2.

The compound represented by Formula 1 may be one of Compound 1 to Compound 5, below:

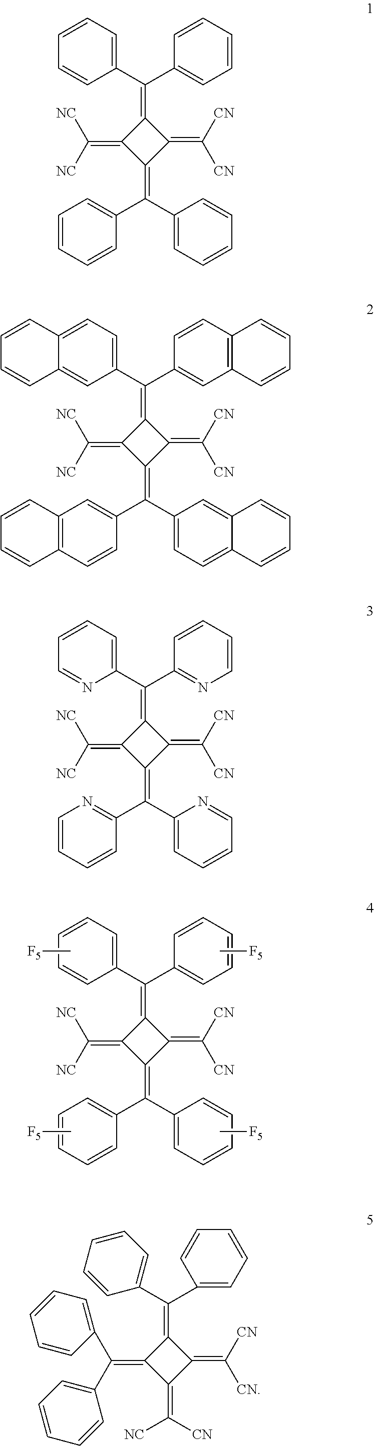

The embodiments may be realized by providing an organic electroluminescence (EL) device including at least one of a hole transport layer and a hole injection layer, wherein the at least one of the hole transport layer and the hole injection layer includes a cyclobutane group-containing compound represented by following Formula 1:

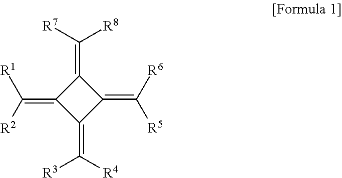

wherein, in Formula 1, R¹ to R⁸ are each independently a substituted or unsubstituted aryl group having 6 to 20 carbon atoms, a substituted or unsubstituted heteroaryl group having 5 to 20 carbon atoms, or a cyano group.

At least one of R¹ to R⁸ may include the substituted aryl group having 6 to 20 carbon atoms or the substituted heteroaryl group having 5 to 20 carbon atoms, and the substituted aryl group having 6 to 20 carbon atoms or the substituted heteroaryl group having 5 to 20 carbon atoms may be substituted with an electron-withdrawing group.

The electron withdrawing group may include a fluorine atom.

Two to four of R¹ to R⁸ may be cyano groups.

The cyclobutane group-containing compound may have a rotational symmetry structure of order 2.

The compound represented by Formula 1 may be one of Compound 1 to Compound 5, below:

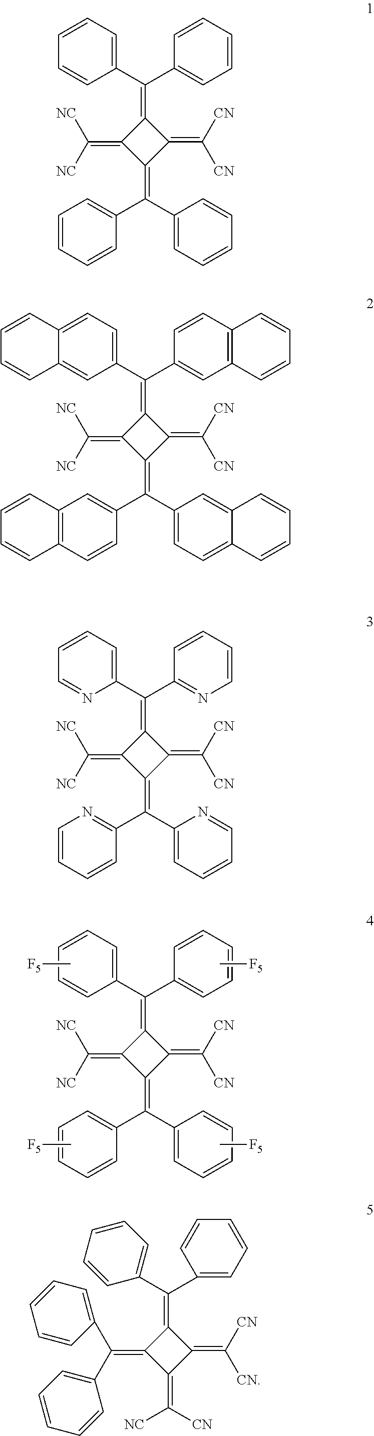

The at least one of the hole transport layer and the hole injection layer may include a host and a dopant, the dopant may include the cyclobutane group-containing compound, and the cyclobutane group-containing compound may be included in the at least one of the hole transport layer and the hole injection layer in an amount of about 0.1 wt % to about 10 wt %, based on a weight of the at least one of the hole transport layer and the hole injection layer.

### BRIEF DESCRIPTION OF THE DRAWING

Features will be apparent to those of skill in the art by describing in detail exemplary embodiments with reference to the attached drawing in which:

FIG. 1 illustrates a schematic diagram of a structure of an organic EL device according to an embodiment.

### DETAILED DESCRIPTION

Example embodiments will now be described more fully hereinafter with reference to the accompanying drawing; however, they may be embodied in different forms and should not be construed as limited to the embodiments set forth herein. Rather, these embodiments are provided so that this disclosure will be thorough and complete, and will fully convey exemplary implementations to those skilled in the art.

In the drawing figure, the dimensions of layers and regions may be exaggerated for clarity of illustration. Like reference numerals refer to like elements throughout.

According to an embodiment, low voltage driving of an organic EL device may be realized by using a cyclobutane group-containing compound (e.g., having a radialene skeleton or core) as a dopant of a hole transport material or a hole injection material. Hereinafter, the cyclobutane group-containing compound (e.g., cyclobutane derivative) and the organic EL device using the same will be explained.

The cyclobutane group-containing compound according to an embodiment may have the radialene skeleton or core. In an implementation, the cyclobutane group-containing compound may be represented by the following Formula 1.

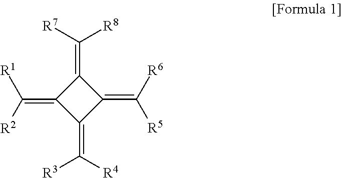

In Formula 1, R¹ to R⁸ may each independently be a substituted or unsubstituted aryl group having 6 to 20 carbon atoms, a substituted or unsubstituted heteroaryl group having 5 to 20 carbon atoms, or a cyano group. In an implementation, the unsubstituted aryl group or heteroaryl group alone may include the above-described number of carbons, and carbons of the substituents on the substituted aryl group or heteroaryl group may not be included in the above-described number of carbons. In the case that the cyclobutane group-containing compound having the above structure is used as a p-type dopants of a hole transport layer or a hole injection layer, an organic EL device including the same may be advantageously driven at a low voltage.

In Formula 1, when at least one of R¹ to R⁸ is the substituted aryl group or the substituted heteroaryl group, an electron-withdrawing group may be substituted on the aryl group or the heteroaryl group. In an implementation, a fluorine atom (F) may be substituted on the aryl group or the heteroaryl group, e.g., the substituted aryl group or substituted heteroaryl group may include a fluorine atom as a substituent. Through the substitution of an electron-withdrawing group (e.g., the fluorine atom) on the aryl group or heteroaryl group, acceptor properties of the cyclobutane group-containing compound may be improved, and the driving voltage of an organic EL device including the cyclobutane group-containing compound may be decreased.

In an implementation, 2 to 4 of R¹ to R⁸ may be cyano groups.

In Formula 1, bonding of 2 to 4 cyano groups as electron-withdrawing-groups at R¹ to R⁸ may help improve the acceptor properties of the cyclobutane group-containing compound according to an embodiment, and the driving voltage of an organic EL device including the same may be decreased.

In an implementation, the cyclobutane group-containing compound according to an embodiment may have a rotational symmetry structure of order 2. For example, the planarity of the molecule of the cyclobutane group-containing compound may be high, and hole transport capability may be improved.

Aromatic amine derivatives may be used as the material of the hole transport layer or the hole injection layer, and may combine with the cyclobutane group-containing compound. In an implementation, the aromatic amine derivatives may include, e.g., triarylamine derivatives, triaryldiamine derivatives, triaryltriamine derivatives, or the like. For example, N-di(1-naphtyl)-N,N-diphenyl-4,4-benzidine, 4,4,4-tris(N-(2-naphtyl)-N-phenylamino)-triphenylamine, or the like may be used.

The cyclobutane group-containing compound according to an embodiment may include, e.g., one of Compound 1 to Compound 5, below.

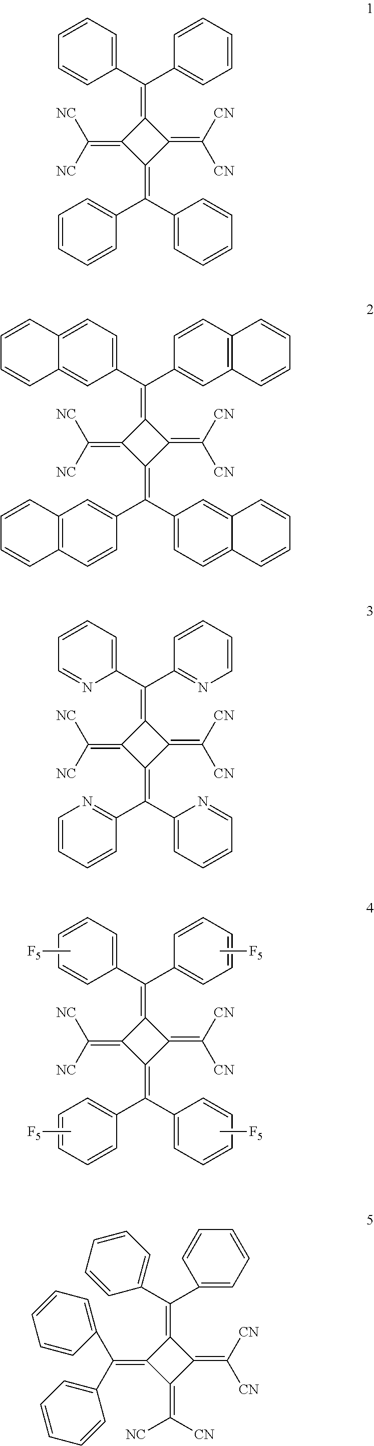

Organic EL Device

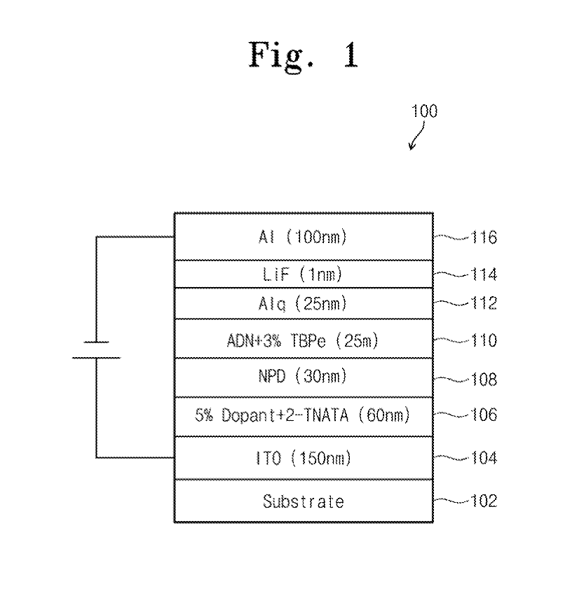

An organic EL device using the cyclobutane group-containing compound as the hole injection material of the organic EL device will be explained referring to FIG. 1.

FIG. 1 illustrates a schematic diagram of the configuration of an organic EL device 100 according to an embodiment. The organic EL device 100 may include, e.g., a substrate 102, an anode 104, a hole injection layer 106, a hole transport layer 108, an emission layer 110, an electron transport layer 112, an electron injection layer 114, and a cathode 116.

In an implementation, the substrate 102 may be a transparent glass substrate, a semiconductor substrate formed of silicon, or the like. In an implementation, the substrate 102 may be a flexible substrate. The anode 104 may be on the substrate 102 and may be formed by using, e.g., indium tin oxide (ITO). In an implementation, the anode 104 may include, e.g., indium zinc oxide (IZO) or the like. The hole injection layer 106 may be on the anode 104. The hole injection layer 106 may be formed by doping the cyclobutane group-containing compound according to an embodiment at about 0.1 wt % to about 10 wt %, e.g., about 1 wt % to about 5 wt %, in a host material including, e.g., 4,4′,4″-tris(N-(2-naphthyl)-N-phenylamino)-triphenylamine (2-TNATA) or the like. The hole transport layer 108 may be on the hole injection layer 106. The hole transport layer 108 may include, e.g., N,N′-di(1-naphthyl)-N,N′-diphenylbenzidine (NPD) or the like. The emission layer 110 may be on the hole transport layer 108. The emission layer 110 may be formed by, e.g., doping 2,5,8,11-tetra-tert-butylperylene (TBPe) or the like in a host material including 9,10-di(2-naphthyl)anthracene (ADN) or the like. The electron transport layer 112 may be on the emission layer 110. The electron transport layer 112 may include, e.g., 8-hydroxyquinoline aluminum (Alq) or the like. The electron injection layer 114 may be on the electron transport layer 112. The electron injection layer 114 may include, e.g., a material including lithium fluoride (LiF). The cathode 116 may be on the electron injection layer 114. The cathode 116 may be formed of, e.g., a metal such as Al. In an implementation, the cathode 116 may be formed of, e.g., a transparent material such as ITO or IZO. The above-described layers and electrodes may be formed by selecting an appropriate layer forming method such as vacuum deposition, sputtering, various coatings, or the like.

In the organic EL device 100 according to an embodiment, the cyclobutane group-containing compound may be used in the hole injection material of the organic EL device in order to help realize the low voltage driving of the organic EL device 100.

In an implementation, the cyclobutane group-containing compound according to an embodiment may be used as a dopants of the hole transport layer 108. Using the cyclobutane group-containing compound in the hole transport material of the organic EL device may also help realize the low voltage driving of the organic EL device 100. When the cyclobutane group-containing compound according to an embodiment is used in the hole transport material of an organic EL device, a doping amount of the cyclobutane group-containing compound may be, e.g., about 0.1 wt % to about 10 wt % or about 1 wt % to about 5 wt %, with respect to a host material such as 2-TNATA or ADN, e.g., with respect to a total weight of the hole transport material.

In an implementation, the cyclobutane group-containing compound according to an embodiment may be doped in one of the hole injection layer 106 or the hole transport layer 108 (e.g., in a host), or in both of the hole injection layer 106 and the hole transport layer 108 (e.g., in a host).

The following Examples and Comparative Examples are provided in order to highlight characteristics of one or more embodiments, but it will be understood that the Examples and Comparative Examples are not to be construed as limiting the scope of the embodiments, nor are the Comparative Examples to be construed as being outside the scope of the embodiments. Further, it will be understood that the embodiments are not limited to the particular details described in the Examples and Comparative Examples.

A synthetic method of the cyclobutane group-containing compound according to an embodiment will be explained herein below. For example, the synthetic method of Compound 1 will be explained referring to the following Reaction Scheme 1.

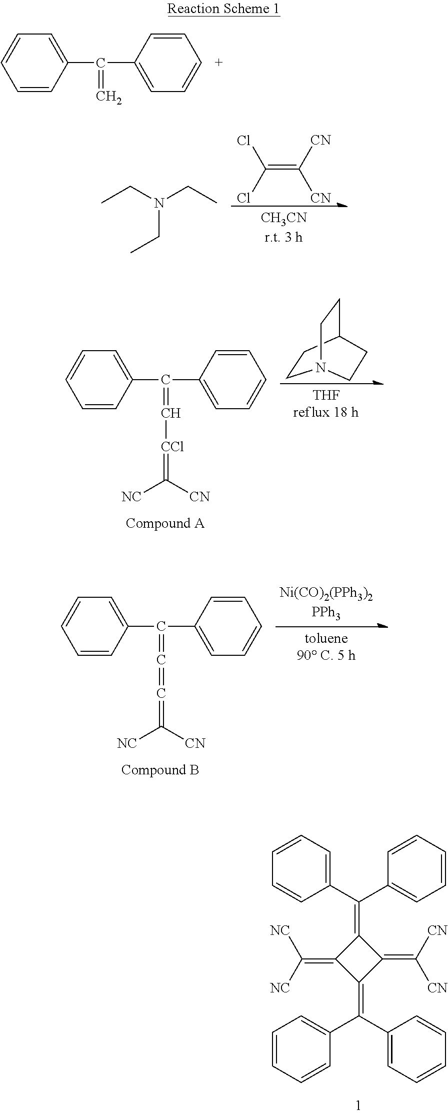

Synthesis of Compound 1

### SYNTHETIC EXAMPLE

A three-necked 300 ml flask was charged with argon gas, and 1.0 g of 1,1-diphenylethylene (Tokyo Chemical Industry Co., Ltd.), 0.6 g of triethylamine (Tokyo Chemical Industry Co., Ltd.) and 100 ml of acetonitrile were added thereto. A solution of 0.88 g of 1,1-dichloro-2,2-dicyanomethylene in 30 ml of acetonitrile was added dropwise thereto. The reaction mixture was stirred at ambient (room) temperature for 3 hours, extracted with toluene and dried with anhydrous magnesium sulfate. Solvent was removed by distillation using a rotary evaporator to obtain Compound A.

Obtained Compound A was dissolved in 100 ml of THF, and 0.7 g of 1-aza-bicyclo[2,2,2]octane (Aldrich Co., LLC.) was added thereto, followed by refluxing for 18 hours while heating. After completing the reaction, the reactant was extracted with toluene and dried with anhydrous magnesium sulfate. Solvent was removed by distillation using a rotary evaporator to obtain Compound B.

A three-necked 300 ml flask was charged with argon gas, and Compound B in 100 ml of toluene was added thereto. 0.2 g of bis(triphenylphosphine)dicarbonyl nickel (Aldrich Co., LLC.) and 1.2 g of triphenylphosphine were added thereto, followed by heating while stirring at 90° C. for 5 hours. The reactant was extracted with toluene and dried with anhydrous magnesium sulfate. Solvent was removed by distillation using a rotary evaporator. The obtained product was separated by using silica gel column chromatography to obtain 0.74 g of a powder.

The molecular weight of the powder was measured by a field diffusion mass spectrometry (FD-MS), and m/z=508 spectrum was obtained for Mw=508. Thus, Compound 1 was identified.

### EXAMPLE

An organic EL device was manufactured by using the above Compound 1 as a p-type dopant. In this example, the substrate 102 was formed by using a transparent glass substrate, the anode 104 was formed using ITO to a thickness of about 150 nm, the hole injection layer 106 having a thickness of about 150 nm was formed by doping about 5 wt % of Compound 1 in 2-TNATA, the hole transport layer 108 was formed using NPD to a thickness of about 30 nm, the emission layer 110 was formed by doping about 3% of TBPe in ADN to a thickness of about 25 nm, the electron transport layer 112 was formed using Alq to a thickness of about 25 nm, the electron injection layer 114 was formed using LiF to a thickness of about 1 nm, and the cathode 116 was formed using Al to a thickness of about 100 nm.

As a Comparative Example, a hole injection layer was formed by doping about 5 wt % of 2,3,5,6-tetrafluoro-7,7,8,8-tetracyanoquinodimethane (F4-TCNQ) in the host material, 2-TNATA, to a thickness of about 60 nm, and an organic EL device was manufactured by conducting the same procedure as described in the above Example.

With respect to the organic EL devices thus manufactured, the driving voltage corresponding to about 10 mA/cm² was measured. The driving voltage of the organic EL device manufactured by forming the hole injection layer by doping F4-TCNQ according to the Comparative Example was 5.2 V, and the driving voltage of the organic EL device manufactured by forming the hole injection layer by doping Compound 1 was 4.3 V. Thus, the driving voltage of the organic EL device including the cyclobutane group-containing compound was quite lower when compared to that according to the Comparative Example.

In an implementation, the cyclobutane group-containing compound may be used as a material in a passive type organic EL device. In an implementation, the cyclobutane group-containing compound of the embodiments may be used in an active type organic EL device, and a decrease of the driving voltage of the active type organic EL device may also be realized.

The organic EL device using the cyclobutane group-containing compound of the embodiments may be used in an organic EL display apparatus or a lighting installation.

By way of summation and review, when using the organic EL device in a display apparatus, low driving voltage of the organic EL device may be desirable, and materials realizing the low driving voltage have been considered. As an organic semiconductor used in the organic EL device, a material doped with a compound having electron-donating properties or a compound having electron-withdrawing properties may change the electrical conductivity thereof.

The embodiments may provide a cyclobutane group-containing compound as a dopant of a hole transport material or a hole injection material, which may be included in an organic electroluminescence device that is driveable at a low voltage.

Various materials for an organic EL device have been considered with a view toward realizing low voltage driving of an organic EL device. The embodiments may provide a dopant of a hole transport material or a hole injection material for realizing the low voltage driving of an organic EL device, and an organic EL device using the same.

The cyclobutane group-containing compound according to an embodiment may include the electron-withdrawing group at the aryl group or the heteroaryl group, and may be used as a hole transport material or a hole injection material having high acceptor properties.

When 2 to 4 among R¹ to R⁸ in the cyclobutane group-containing compound according to an embodiment are the cyano groups, the cyclobutane group-containing compound may be used as a hole transport material or a hole injection material having high acceptor properties.

The cyclobutane group-containing compound according to an embodiment may be used as a hole transport material or a hole injection material having high hole transporting properties.

The organic EL device according to an embodiment may include the hole transport layer or the hole injection layer containing the cyclobutane group-containing compound represented by Formula 1, and may be driven at a low voltage.

The cyclobutane group-containing compound according to an embodiment may include the electron-withdrawing group at the aryl group or the heteroaryl group, and may be used as a hole transport material or a hole injection material having high acceptor properties.

When 2 to 4 among R¹ to R⁸ in the cyclobutane group-containing compound are the cyano groups in the organic EL device according to an embodiment, the acceptor properties of a hole transport material or a hole injection material may be improved.

The organic EL device according to an embodiment may exhibit improved hole transporting properties of the hole transport material and/or the hole injection material.

According to an embodiment, a hole transport material or a hole injection material for an organic EL device realizing low voltage driving, and an organic EL device using the same may be provided.

Example embodiments have been disclosed herein, and although specific terms are employed, they are used and are to be interpreted in a generic and descriptive sense only and not for purpose of limitation. In some instances, as would be apparent to one of ordinary skill in the art as of the filing of the present application, features, characteristics, and/or elements described in connection with a particular embodiment may be used singly or in combination with features, characteristics, and/or elements described in connection with other embodiments unless otherwise specifically indicated. Accordingly, it will be understood by those of skill in the art that various changes in form and details may be made without departing from the spirit and scope of the present invention as set forth in the following claims.

What is claimed is:

1. A cyclobutane group-containing compound represented by following Formula 1: 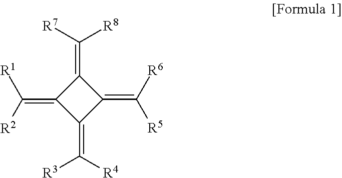
wherein, in Formula 1, R¹ to R⁸ are each independently a substituted or unsubstituted aryl group having 6 to 20 carbon atoms, a substituted or unsubstituted heteroaryl group having 5 to 20 carbon atoms, or a cyano group.

2. The cyclobutane group-containing compound as claimed in claim 1, wherein:
at least one of R¹ to R⁸ includes the substituted aryl group having 6 to 20 carbon atoms or the substituted heteroaryl group having 5 to 20 carbon atoms, and
the substituted aryl group having 6 to 20 carbon atoms or the substituted heteroaryl group having 5 to 20 carbon atoms is substituted with an electron-withdrawing group.

3. The cyclobutane group-containing compound as claimed in claim 2, wherein the electron withdrawing group includes a fluorine atom.

4. The cyclobutane group-containing compound as claimed in claim 2, wherein two to four of R¹ to R⁸ are cyano groups.

5. The cyclobutane group-containing compound as claimed in claim 1, wherein two to four of R¹ to R⁸ are cyano groups.

6. The cyclobutane group-containing compound as claimed in claim 1, wherein the cyclobutane group-containing compound has a rotational symmetry structure of order 2.

7. The cyclobutane group-containing compound as claimed in claim 1, wherein the compound represented by Formula 1 is one of Compound 1 to Compound 5, below:

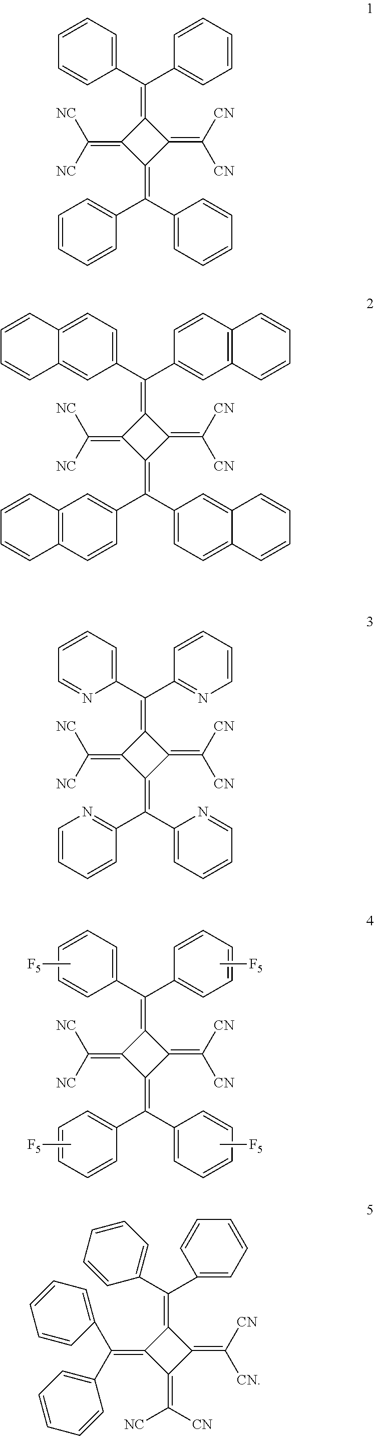

8. An organic electroluminescence (EL) device, comprising at least one of a hole transport layer and a hole injection layer, wherein the at least one of the hole transport layer and the hole injection layer includes a cyclobutane group-containing compound represented by following Formula 1: 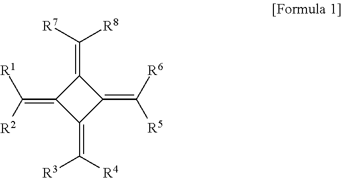
wherein, in Formula 1, R¹ to R⁸ are each independently a substituted or unsubstituted aryl group having 6 to 20 carbon atoms, a substituted or unsubstituted heteroaryl group having 5 to 20 carbon atoms, or a cyano group.

9. The organic EL device as claimed in claim 8, wherein:
at least one of R¹ to R⁸ includes the substituted aryl group having 6 to 20 carbon atoms or the substituted heteroaryl group having 5 to 20 carbon atoms, and the substituted aryl group having 6 to 20 carbon atoms or the substituted heteroaryl group having 5 to 20 carbon atoms is substituted with an electron-withdrawing group.

10. The organic EL device as claimed in claim 9, wherein the electron withdrawing group includes a fluorine atom.

11. The organic EL device as claimed in claim 9, wherein two to four of R¹ to R⁸ are cyano groups.

12. The organic EL device as claimed in claim 8, wherein two to four of R¹ to R⁸ are cyano groups.

13. The organic EL device as claimed in claim 8, wherein the cyclobutane group-containing compound has a rotational symmetry structure of order 2.

14. The organic EL device as claimed in claim 8, wherein the compound represented by Formula 1 is one of Compound 1 to Compound 5, below:

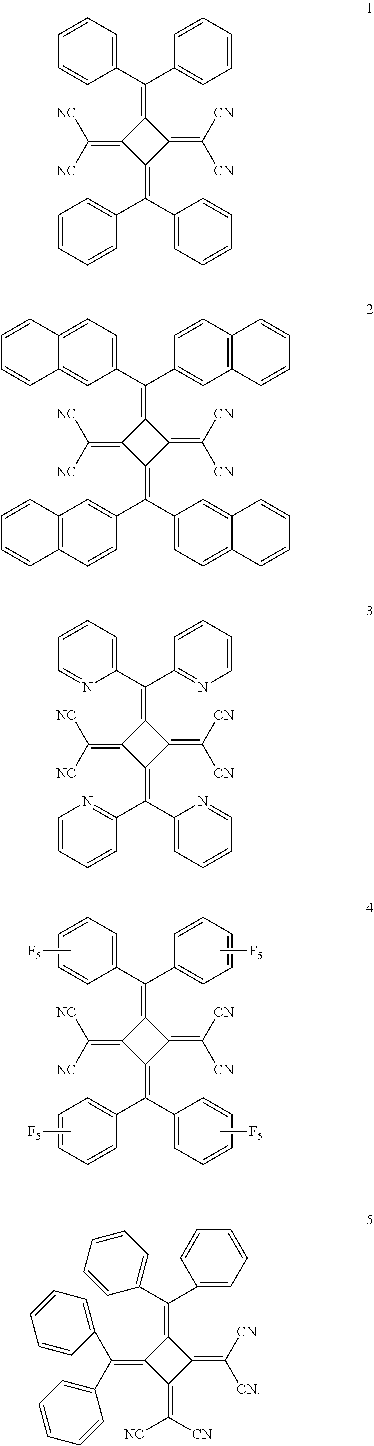

15. The organic EL device as claimed in claim 8, wherein:
the at least one of the hole transport layer and the hole injection layer includes a host and a dopant,
the dopant includes the cyclobutane group-containing compound, and
the cyclobutane group-containing compound is included in the at least one of the hole transport layer and the hole injection layer in an amount of about 0.1 wt % to about 10 wt %, based on a weight of the at least one of the hole transport layer and the hole injection layer.
